SEQUENCE LISTING (4 sequences)

SEQ ID NO: 1 - DNA, 23 bp, Artificial Sequence
  PCR forward primer
ttccaaaagccacactcaaacac

SEQ ID NO: 2 - DNA, 25 bp, Artificial Sequence
  PCR reverse primer
tggtgatgttcacagtctctgaaga

SEQ ID NO: 3 - DNA, 23 bp, Artificial Sequence
  V158 specific probe
ttactcccaaaaagccccctgca

SEQ ID NO: 4 - DNA, 22 bp, Artificial Sequence
  F158 allele specific probe
tactcccaacaagccccctgca

(19) United States
(12) Patent Application Publication
(10) Pub. No.: US 2013/0064838 A1
(43) Pub. Date: Mar. 14, 2013

(54) ENHANCED DEATH RECEPTOR AGONISTS

(71) Applicants: Jonathan David Graves, Belmont, MA (US); Jennifer Joy Kordich, Sammamish, WA (US); Susan Ellen Cottrell, Seattle, WA (US); Chang-Pin Huang, Shoreline, WA (US)

(72) Inventors: Jonathan David Graves, Belmont, MA (US); Jennifer Joy Kordich, Sammamish, WA (US); Susan Ellen Cottrell, Seattle, WA (US); Chang-Pin Huang, Shoreline, WA (US)

(73) Assignee: AMGEN INC., Thousand Oaks, CA (US)

(21) Appl. No.: 13/698,040

(22) PCT Filed: May 13, 2011
(86) PCT No.: PCT/US2011/036521

§ 371 Date: Nov. 14, 2012

Related U.S. Application Data
(60) Provisional application No. 61/345,003, filed on May 14, 2010.

(51) Int. Cl. C07K 16/28; A61P 35/00; C12N 5/09; C12Q 1/68; A61K 39/395

(52) U.S. Cl. 424/174.1; 435/6.11; 530/389.1; 530/387.3; 435/366

(57) ABSTRACT

Methods and compositions for treatment of cancer in a human patient comprising administering a therapeutically effective amount of an Fc-polypeptide agonist of DR5 having high-affinity to FCGR3A. Methods of making the Fc-polypeptides are provided.

### CROSS REFERENCE TO RELATED APPLICATIONS

This application hereby claims the benefit of U.S. Provisional Application Ser. No. 61/345,003 filed May 14, 2010, the entire disclosure of which is relied upon and incorporated by reference.

### FIELD OF THE INVENTION

The present invention relates to compositions and methods for improving the clinical benefit obtained from death receptor agonists in the treatment of cancer.

### BACKGROUND OF THE INVENTION

The interaction between death receptor 5 (DR5) or death receptor 4 (DR4) and their ligand, TRAIL (TNF-Receptor Apoptosis Inducing Ligand), plays a key role in the induction of apoptosis of cells. TRAIL, also known as Apo2 ligand, is a homotrimeric ligand that interacts with four members of the TNF-receptor superfamily (TRAIL receptors 1 to 4), as well as with the soluble osteoprotegerin (“OPG”) receptor. Binding of TRAIL to DR4 or DR5 at the surface of a sensitive cancer cell triggers an apoptotic cascade that first requires a process of cross-linking to exert its effect. Cross-linking is thought to induce clustering of the death receptors and activation of the signaling cascade resulting in the induction of apoptosis. This process is mediated in vivo by the Fcγ receptor IIIA (“FCGR3A”) which is expressed mainly on natural killer (NK) cells and, to a lesser extent, on macrophages. Upon clustering, intracellular proteins are recruited to the intracellular death domain of the receptor, forming a signaling complex. In turn, certain intracellular caspases are then recruited to the complex where they autoactivate and, in turn, activate additional caspases and the intracellular apoptosis cascade leading to cell death. Recognizing the therapeutic potential of apoptosis in the treatment of cancer, researchers have developed a variety of DR5 and DR4 agonistic antibodies. What is needed in the art is a means to further improve the clinical benefit of DR agonists.

### SUMMARY OF THE INVENTION

In one aspect the present invention is directed to agonistic high-affinity Fc-polypeptides, such as antibodies or peptibodies, that specifically bind to cells expressing human DR4 and/or DR5 and induce apoptosis in apoptosis induction sensitive cells, such as human cancer cells or virus infected cells. The Fc of the Fc-polypeptide of the invention has increased affinity to FCGR3A receptor relative to the native Fc. In some embodiments the Fc-polypeptide is a fully-human IgG1 antibody. In some embodiments the Fc is a afucosylated or modified at position 332 (per the EU index of Kabat).

The present invention also includes a method of inhibiting growth of a human cancer cell in vitro or in vivo by administering an effective amount of a high-affinity Fc-polypeptide of the invention. In some embodiments the high-affinity Fc-polypeptide is administered to a human patient comprising the cancer. In some embodiments the cancer is non-small cell lung cancer. In some embodiments the Fc-polypeptide is administered as a monotherapy while in other embodiments it is administered with one or more chemotherapeutic agents such as carboplatin in combination with paclitaxel. In a further aspect, the present invention is directed to a means of selecting a human cancer patient with increased statistical likelihood of obtaining a clinical benefit from treatment with a high-affinity Fc-polypeptide of the invention. The selected patient is heterozygous or homozygous for the FCGR3A F158 allele. Compositions and methods for genotyping the FCGR3A polymorphism in a human genomic DNA sample are also provided.

### DETAILED DESCRIPTION OF INVENTION

The present invention relates, in part, to high-affinity Fc-polypeptides that agonize DR5 (or DR4) in that they specifically bind to DR5 (and/or DR4) on human cells and induce apoptosis in cells sensitive to DR5 (and/or DR4) mediated apoptotic induction. Some high-affinity antibodies have been reported to improve ADCC (antibody-dependent cell-mediated cytotoxicity). Surprisingly, however, the anti-cancer activity of the apoptotic Fc-polypeptides of the present invention is also enhanced.

The section headings are used herein for organizational purposes only, and are not to be construed as in any way limiting the subject matter described. The disclosure of all patents, patent applications, and other documents cited herein are hereby expressly incorporated by reference in their entirety. Unless specific definitions are provided, the nomenclature utilized in connection with, and the laboratory procedures and techniques of analytical chemistry, synthetic organic chemistry, and medicinal and pharmaceutical chemistry described herein are those well known and commonly used in the art. Standard techniques may be used for chemical syntheses, chemical analyses, pharmaceutical preparation, formulation, and delivery, and treatment of patients.

### Definitions

The terms used throughout this specification are defined as follows, unless otherwise limited in specific instances.

The term “afucosylation” or “afucosylated” in the context of an Fc refers to a substantial lack of a fucose covalently attached, directly or indirectly, to residue 297 of the human IgG1 Fc numbered according to the EU index (Kabat et al., Sequences of Proteins of Immunological Interest, 5th Ed. Public Health Service, National Institutes of Health, Bethesda, Md. (1991)), or the corresponding residue in non-IgG1 or non-human IgG1 immunoglobulins. Thus, in a composition comprising a plurality of afucosylated Fc-polypeptides at least 70% of the Fc-polypeptides will be not be fucosylated, directly or indirectly (e.g., via intervening sugars) at residue 297 of the Fc, and in some embodiments at least 80%, 85%, 90%, 95%, or 99% will not be fucosylated, directly or indirectly at residue 297 of the Fc.

The term “agonist” or “agonistic” or “agonize” in the context of an Fc-polypeptide, refers to DR4 and/or DR5 mediated induction of apoptosis in an apoptosis sensitive mammalian cancer cell, such as a human cancer cell, which expresses DR4 and/or DR5 on the cell surface. An exemplary sensitive human cancer cell is Co1o205 (ATCC CCL-222). A DR5 agonist will induce apoptosis via DR5, a DR4 agonist will induce apoptosis via DR4, a dual DR5/DR4 agonist (e.g., TRAIL ligand or a bispecific agonistic anti-DR4/DR5 antibody) is able to induce apoptosis through both DR4 and/or DR5. Whether apoptotic induction is mediated via DR5 and/or DR4 can be determined using methods and reagents known in the art. Thus, for example, apoptosis sensitive DR specific cell lines are known in the art. An exemplary DR5(+)/DR4(−) cell line is WM35 (ATCC CRL-2807). An exemplary DR5(−)/DR4(+) cell line is ST486 (ATCC CRL 1647).

The term “antibody” includes reference to isolated forms of both glycosylated and non-glycosylated immunoglobulins of any isotype or subclass, including any combination of: 1) human (e.g., CDR-grafted), humanized, and chimeric antibodies, 2) monospecific (e.g., DR5 or DR4) or multi-specific antibodies (e.g., DR4 and DR5), and 3) monoclonal or polyclonal antibodies, irrespective of whether such antibodies are produced, in whole or in part, via immunization, through recombinant technology, by way of in vitro synthetic means, or otherwise. Thus, the term “antibody” is inclusive of antibodies that are prepared, expressed, created or isolated by recombinant means, such as (a) antibodies isolated from an animal (e.g., a mouse) that is transgenic for human immunoglobulin genes or a hybridoma prepared therefrom, (b) antibodies isolated from a host cell transfected to express the antibody (e.g., from a transfectoma), (c) antibodies isolated from a recombinant, combinatorial antibody library, and (d) antibodies prepared, expressed, created or isolated by any other means that involve splicing of immunoglobulin gene sequences to other DNA sequences. Antibodies are also inclusive of antibody fragments such as Fab, F(ab′)₂, scFv (single-chain Fv), and derivatives such as diabodies. In some embodiments the antibodies of the present invention are monoclonal antibodies, such as humanized or fully-human monoclonal antibodies. Typically, antibodies of the present invention will be IgG1 or IgG2 subclass antibodies. The antibody may bind its target with a Kd of less than about 10 nM, 5 nM, 1 nM, or 500 pM.

The terms “derivation” or “derivatives” refer to modification of an Fc-polypeptide (such as an antibody) and/or chemotherapeutic agent by covalently linking it, directly or indirectly, so as to modify such characteristics as half-life, bioavailability, immunogenicity, solubility, or hypersensitivity properties, while retaining its therapeutic benefit. Derivatives can be made by glycosylation, pegylation, and lipidation, or by protein conjugation. Exemplary derivitizing agents include a linear polymer (e.g., polyethylene glycol (PEG), polylysine, dextran, etc.); a branched-chain polymer (See, for example, U.S. Pat. No. 4,289,872 to Denkenwalter et al., issued Sep. 15, 1981; U.S. Pat. No. 5,229,490 to Tam, issued Jul. 20, 1993; WO 93/21259 by Frechet et al., published 28 Oct. 1993); a lipid or liposome; a cholesterol group (such as a steroid); a carbohydrate or oligosaccharide.

The terms “DR4” or “death receptor 4” or “TRAIL-R1” or “TR-1” refer to the 468 amino acid polypeptide set forth in SEQ ID NO: 2 of U.S. Pat. No. 6,342,363 (incorporated herein by reference) as well as related native (i.e., wild-type) human polypeptides such as allelic variants, splice variants, and mature forms of the polypeptide (i.e., lacking a leader sequence).

The term “DR5” or TRAIL-R” or “Apo-2” or “TR-2” or “TRAIL Receptor-2” refer to the 440 amino acid polypeptide set forth in SEQ ID NO: 2 of U.S. Pat. No. 7,528,239 as well as related native (i.e., wild-type) human polypeptides such as allelic variants or splice variants such as, but not limited to, the 411 amino acid isoform set forth in SEQ ID NO: 1 in U.S. Pat. No. 6,342,369, and at SEQ ID NO: 2 of U.S. Pat. No. 6,743,625 (each patent incorporated herein by reference), including mature forms of the polypeptide (i.e., lacking a leader sequence).

The terms “DR5 agonist” refers to a molecule that specifically binds to native human DR5 on cells expressing it and via this receptor triggers an apoptotic cascade resulting in a statistically significant increase in cell death (i.e., apoptosis) as measured in at least one DR5 agonist sensitive cell line (including, but not limited to, the human colon carcinoma cell line Colo 205, or the human lung carcinoma cell line H2122). The term “DR4 agonist” refers to a molecule that specifically binds to native human DR4 on cells expressing it and via this receptor triggers an apoptotic cascade resulting in a statistically significant increase in cell death (i.e., apoptosis) as measured in at least one DR4 agonist sensitive cell line (including, but not limited to, the human colon carcinoma cell line Colo 205, or the human lung carcinoma cell line H2122). In certain embodiments, the DR5 and/or DR4 agonist is an Fc-polypeptide such as an antibody, peptibody, human TRAIL (see, U.S. Pat. Nos. 6,284,236; 6,998,116, both of which are incorporated herein by reference), Fc-human TRAIL ligand fusion, or a non-proteinaceous, non-polymeric molecule of less than about 1000 Daltons (a “small molecule”) as for example the DR5 small molecule agonist of U.S. Ser. No. 11/866,162 (Srivastava et al.) or an Fc covalently bound to a DR5 small molecule.

The terms “effective amount” or “therapeutically effective amount” refer to a quantity and/or concentration of an Fc-polypeptide that when administered ex vivo (by contact with a cancer cell from a human patient) or in vivo (by administration into a human patient) for treatment of a DR5 (and/or DR4) sensitive cancer either alone (i.e., as a monotherapy) or in combination with a chemotherapeutic agent (i.e., as a combination therapy) yields a statistically significant inhibition of cancer progression. As used herein, the terms “treatment” or, “inhibit,” “inhibiting” or “inhibition” of cancer refers to at least one of: a statistically significant decrease in the rate of tumor growth, a cessation of tumor growth, or a reduction in the size, mass, metabolic activity, or volume of the tumor, as measured by standard criteria such as, but not limited to, the Response Evaluation Criteria for Solid Tumors (RECIST), or a statistically significant increase in progression free survival (PFS) or overall survival (OS).

The term “Fc” in the context of an “Fc-polypeptide” refers to the Fc (fragment crystallizable) of an immunoglobulin that specifically binds to human FCGR3A. An Fc is generally naturally-occurring (“native”) human IgG1 Fc but also includes truncated forms of IgG1 Fc (“truncated Fc”) that specifically bind to FCGR3A, or variants of naturally-occurring IgG1 Fc (“Fc variants”) made by substitution, deletion, or addition of amino acid residues wherein the variant Fc specifically binds to FCGR3A. A truncated Fc can be at least 80%, 85%, 90%, 95%, 96%, 97%, 98%, or 99% of the full-length Fc. The number of substitutions, deletions, or additions of a truncated Fc or of an Fc variant can be up to 1, 2, 3, 4, 5, 6, 7, 8, 9, 10, 15, or 20. Specific binding of a truncated Fc or Fc variant to FCGR3A is generally at least 80%, 85%, 90% or 95% of native Fc specific binding.

The terms “FCGR3A” or “CD16a” “Fey receptor IIIA” or “FcγRIIIA” means the human Fc receptor of the same designation. A bi-allelic polymorphism of the human IgG receptor FcγRIIIA (CD16a) termed “F158V” can be distinguished by virtue of the presence of the amino acid valine (V) or phenylalanine (F) at the locus identified at the National Center for Biotechnology Information (NCBI) Single Nucleotide Polymorphism (SNP) database at cluster report rs396991. These two alleleic forms are commonly referred to in the literature and herein as “valine158” or “V158” for the polymorphism having the residue valine at the rs396991 SNP locus of human FcγRIIIA, and “phenylalanine 158” or “F158” for the polymorphism having the residue phenylalanine at the rs396991 SNP locus of human FcγRIIIA. See also, Leppers-van de Straat et al., J. Immunological Methods, 242: 127-132 (2000) and Ravetch and Perussia, J. Exp. Med., 170:481-497 (1989).

The term “Fc-polypeptide” refers to the product of a covalent attachment between an Fc and at least one polypeptide that specifically binds to DR4 and/or DR5. The fusion of Fc and polypeptide may be via a direct covalent bond (via a peptide bond) or indirect covalent bond (via a man-made chemical linker). Typically, the Fc-polypeptide is an agonistic Fc-polypeptide. Exemplary Fc-polypeptides include antibodies, peptibodies (WO 2000/24782, incorporated herein by reference), antibodies conjugated to targeting peptides (see, e.g., U.S. Pat. No. 7,521,425, incorporated herein by reference) or a cytotoxin, or an Fc-human TRAIL ligand fusion. In some embodiments, the Fc-polypeptide is bivalent. In some embodiments, the Fc-polypeptide is bivalent and bispecific. In some embodiments, the Fc-polypeptide is a homodimer comprising two IgG1 Fcs and in some embodiments the Fc-polypeptide is a heterodimer comprising one IgG1 Fc and one non-IgG1 Fc. In some embodiments the homodimer and heterodimers are fully human antibodies.

The term “high-affinity” in the context of an Fc-polypeptide, means that the Fc is modified or constructed such that it specifically binds to human FCGR3A expressed by a native cell (e.g., a human NK cell) that is homozygous for the F158 allele with at least the same affinity as at least one of: an identical but afucosylated human Fc-polypeptide (e.g., an antibody), or an identical human Fc-polypeptide comprising a modification to increase FCGR3A affinity at residue 332 (per EU index of Kabat; see, U.S. Pat. No. 7,317,091 and/or U.S. Pat. No. 7,662,925) such as a isoleucine to glutamic acid substitution. Generally, a high-affinity Fc-polypeptide specifically binds to human FCGR3A with at least the same affinity as a native fucosylated Fc-polypeptide specifically binds to human FCGR3A expressed by a native cell homozygous for the V158 allele. Means to measure binding affinity are known in the art and include but are not limited to competition assays such as an A1phaLISA™ (Perkin Elmer, Waltham, Mass. USA) ELISA assay. See, Poulsen, J., et al. 2007. J. Biomol Screen. 12:240; Cauchon, E., et al. 2009. Anal Biochem.

The term “host cell” refers to a cell that can be used to express a nucleic acid, e.g., a nucleic acid of the present invention. A host cell can be a prokaryote, for example, E. coli, or it can be a eukaryote, for example, a single-celled eukaryote (e.g., a yeast or other fungus), a plant cell (e.g., a tobacco or tomato plant cell), an animal cell (e.g., a human cell, a monkey cell, a hamster cell, a rat cell, a mouse cell, or an insect cell) or a hybridoma. Examples of host cells include Chinese hamster ovary (CHO) cells or their derivatives such as Veggie CHO and related cell lines which grow in serum-free media (see Rasmussen et al., Cytotechnology 28: 31, 1998) or CHO strain DX-B11, which is deficient in DHFR (see Urlaub et al., Proc. Natl. Acad. Sci. USA 77: 4216-4220, 1980).

The term “human antibody” or “fully human antibody” refers to an antibody in which both the constant regions and the framework consist of fully or substantially human sequences such that the human antibody typically elicits substantially no immunogenic reaction against itself when administered to a human and, preferably, elicits no detectable immunogenic response. Thus, the defined terms contemplate minor amino acid modifications (often no more than 1, 2, 3, or 4 amino acid substitutions, additions, or deletions) made relative to a native human antibody sequence to allow, for example, for improved formulation or manufacturability (e.g., removal of unpaired cysteine residues).

The term “humanized antibody” refers to an isolated antibody in which substantially all of the constant region is derived from or corresponds to human immunoglobulins, while all or part of one or more variable regions is derived from another species, for example a mouse.

The term “isolated” refers to a compound that: (1) is substantially purified (e.g., at least 60%, 70%, 80%, or 90%) away from cellular components with which it is admixed in its expressed state such that it is the predominant species present, (2) is conjugated to a polypeptide or polynucleotide or other moiety to which it is not linked in nature, (3) does not occur in nature as part of a larger polypeptide or polynucleotide sequence, (4) is combined with other chemical or biological agents having different specificities in a well-defined composition, or (5) comprises a human engineered sequence not otherwise found in nature.

The terms “monoclonal antibody” or “monoclonal antibody composition” refers to a preparation of isolated antibody molecules of single molecular composition (notwithstanding minor heterogeneities resulting from, for example, post-translational modification such as glycosylation and/or signal sequence cleavage), typically encoded by the same nucleic acid molecule. A monoclonal antibody composition displays a single binding specificity and affinity for a particular epitope. In certain embodiments, monoclonal antibodies are produced by a single hybridoma or other cell line (e.g., a transfectoma), or by a transgenic mammal. The term “monoclonal” is not limited to any particular method for making an antibody.

The term “naturally occurring” or “native” when used in connection with biological materials such as nucleic acid molecules, polypeptides, host cells, and the like, refers to those which are found in nature and not modified by a human.

The terms, “nucleic acid” and “polynucleotide” refer to a deoxyribonucleotide or ribonucleotide polymer, or chimeras thereof, and unless otherwise limited, encompasses the complementary strand of the referenced sequence. A nucleic acid sequence is “operably linked” to a regulatory sequence if the regulatory sequence affects the expression (e.g., the level, timing, or location of expression) of the nucleic sequence. A “regulatory sequence” is a nucleic acid that affects the expression (e.g., the level, timing, or location of expression) of a second nucleic acid. Thus, a regulatory sequence and a second sequence are operably linked if a functional linkage between the regulatory sequence and the second sequence is such that the regulatory sequence initiates and mediates transcription of the DNA sequence corresponding to the second sequence. Examples of regulatory sequences include promoters, enhancers and other expression control elements (e.g., polyadenylation signals). Further examples of regulatory sequences are described in, for example, Goeddel, 1990, Gene Expression Technology: Methods in Enzymology 185, Academic Press, San Diego, Calif. and Baron et al., Nucleic Acids Res. 23: 3605-3606, 1995.

The terms “peptide,” “polypeptide” and “protein” are used interchangeably throughout and refer to a molecule comprising two or more amino acid residues joined to each other by peptide bonds. The terms “polypeptide”, “peptide” and “protein” are also inclusive of modifications including, but not limited to, glycosylation, lipid attachment, sulfation, gamma-carboxylation of glutamic acid residues, hydroxylation and ADP-ribosylation.

The term “peptibody” refers to a peptide that specifically binds to a designated target and in which the peptide is covalently bonded (e.g., via a peptide bond) to the N- or C-terminus of an antibody Fc such as a human IgG1 Fc. The production of peptibodies is generally described in PCT publication WO 00/24782, published May 4, 2000, incorporated herein by reference. Exemplary peptides may be generated by any of the methods set forth herein, such as carried in a peptide library (e.g., a phage display library), generated by chemical synthesis, derived by digestion of proteins, or generated using recombinant DNA techniques.

The terms “peptibody fragment” or “antibody fragment” refers to a peptide of a peptibody or antibody which comprises less than a complete intact peptibody or antibody but retains the ability to specifically bind to its target molecule (i.e., human DR5 or human DR4). Exemplary fragments includes F(ab) or F(ab′)2 fragments. Such a fragment may arise, for example, from a truncation at the amino terminus, a truncation at the carboxy-terminus, and/or an internal deletion of a residue(s) from the amino acid sequence. Fragments may result from alternative RNA splicing or from in vivo or in vitro protease activity. Such fragments may also be constructed by chemical peptide synthesis methods, or by modifying a polynucleotide encoding an antibody or peptibody.

The terms “polynucleotide,” “oligonucleotide” and “nucleic acid” are used interchangeably throughout and include DNA molecules (e.g., cDNA or genomic DNA), RNA molecules (e.g., mRNA), and hybrids thereof. The nucleic acid molecule can be single-stranded or double-stranded.

The term “specifically binds” refers to the ability of an Fc-polypeptide of the present invention, under specific binding conditions, to bind to a target (e.g., human DR5, human DR4, or human FCGR3A) such that its affinity is at least 10 times as great, but optionally 50 times as great, 100, 250 or 500 times as great, or even at least 1000 times as great as the average affinity of the same molecule to a collection of random peptides or polypeptides of sufficient statistical size. An Fc-polypeptide need not bind exclusively to a single target molecule but may specifically bind to a non-target molecule due to similarity in structural conformation between the target and non-target (e.g., paralogs or orthologs). Those of skill will recognize that specific binding to a molecule having the same function in a different species of animal (i.e., ortholog) or to a molecule having a substantially similar epitope as the target molecule (e.g., a paralog) is within the scope of the term “specific binding” which is determined relative to a statistically valid representation of unique non-targets (e.g., random polypeptides). Thus, an anti-DR5 Fc-polypeptide of the invention may specifically bind to more than one distinct species of target molecule, such as specifically binding to both DR5 and DR4. Solid-phase ELISA immunoassays can be used to determine specific binding. Generally, specific binding proceeds with an association constant of at least about 1×10⁷ M⁻¹, and often at least 1×10⁸ M⁻¹, 1×10⁹M⁻¹, or, 1×10¹⁰ M⁻¹.

The term “vector” refers to a nucleic acid used in the introduction of a polynucleotide of the present invention into a host cell. Vectors are often replicons. Expression vectors permit transcription of a nucleic acid inserted therein when present in a suitable host cell or under suitable in vitro conditions.

### Fc-Polypeptides

The present invention provides Fc-polypeptides with enhanced anti-cancer activity. Structurally, these Fc-polypeptides combine an enhanced affinity (a “high-affinity”) to human FCGR3A with a DR4 and/or DR5 agonistic function. As agonists the Fc-polypeptides of the invention induce apoptosis of sensitive human cancer cells by specifically binding to, and mediating apoptosis through, human DR4 and/or human DR5.

The present invention thus provides agonistic high-affinity Fc-polypeptides wherein the Fc is afucosylated to increase affinity to human FCGR3A. In some embodiments the Fc is an afucosylated fully human IgG1 Fc. In some embodiments the Fc-polypeptide is an afucosylated fully human IgG1 monoclonal antibody. In some embodiments the afucosylated fully human IgG1 monoclonal antibody specifically binds to human DR5 and/or human DR4. Thus in some embodiments the afucosylated fully human IgG1 monoclonal antibody is a bispecific antibody that specifically binds to human DR5 and human DR4. In some embodiments the Fc-polypeptide is a fully human IgG1 monoclonal antibody that specifically binds to human DR5 but does not specifically bind to (i.e., does not cross-react with) human DR4. In some embodiments the Fc-polypeptide specifically binds to human DR4 but does not specifically bind to (i.e., does not cross react with) human DR5. Methods of creating afucosylated or antibodies or Fc-fusion peptides are known in the art and include, but are not limited to, recombinant expression using genetic (e.g., siRNA) or chemical means to inhibit cellular fucosyl transferase function or expression, using host cells missing the gene for fucosyl transferase (e.g., fut8 knock-outs), or defucosylating the Fc by in vitro chemical or enzymatic means. See, e.g., U.S. Pat. No. 6,946,292, incorporated herein by reference. Those of skill will recognize that compositions comprising a plurality of high-affinity Fc-polypeptides of the invention need not be 100% afucosylated to exhibit enhanced anti-cancer activity but generally comprise at least 50%, 60%, 70%, 75%, 80%, 85%, 90%, 95%, 97%, 98%, or 99% afucosylated Fc-polypeptides.

The present invention also provides an agonistic high-affinity Fc-polypeptide wherein the Fc comprises at least one amino acid substitution that yields enhanced FCGR3A affinity as described in U.S. Pat. No. 7,317,091 (incorporated herein by reference). In some embodiments, the Fc comprising an aforementioned amino acid substitution is a human IgG1 Fc. In some embodiments the Fc-polypeptide comprising at least one amino acid substitutions to enhance FCGR3A binding is a fully human IgG1 monoclonal antibody. In some embodiments the fully human IgG1 monoclonal antibody specifically binds to human DR5 and/or human DR4. Thus in some embodiments the fully human IgG1 monoclonal antibody is a bispecific antibody that specifically binds to human DR5 and human DR4. In some embodiments the Fc-polypeptide is a fully human IgG1 monoclonal antibody that specifically binds to human DR5 but does not specifically bind to (i.e., does not cross-react with) human DR4. In some embodiments the Fc-polypeptide specifically binds to human DR4 but does not specifically bind to (i.e., does not cross react with) human DR5. In some embodiments, the Fc comprises a substitution at, at least one of, residues: 230, 233, 234, 235, 239, 240, 243, 264, 266, 272, 274, 275, 276, 278, 302, 318, 324, 325, 326, 328, 330, 332, and 335, wherein the numbering of the residues in the Fc region is that of the EU index as in Kabat. In some embodiments, the Fc comprises at least one amino acid substitution selected from the group consisting of: P230A, E233D, L234E, L234Y, L234I, L235D, L235S, L235Y, L235I, S239D, S239E, S239N, S239Q, S239T, V240I, V240M, F243L, V264I, V264T, V264Y, V266I, E272Y, K274T, K274E, K274R, K274L, K274Y, F275W, N276L, Y278T, V3021, E318R, S324D, S324I, S324V, N325T, K326I, K326T, L328M, L328I, L328Q, L328D, L328V, L328T, A330Y, A330L, A330I, I332D, 1332E, I332N, I332Q, T335D, T335R, and T335Y wherein the letter preceding the number represents in one-letter amino acid code the substitution residue, the number indicates the residue of the Fc numbered per the EU index as in Kabat and the letter following the number indicates the native residue. In some embodiments, the high-affinity Fc-polypeptide comprises both an afucosylated Fc and an amino acid substitution to enhance FCGR3A affinity as described above. In some embodiments, the Fc of the Fc-polypeptide comprises 1, 2, 3, 4, 5, 6, 7, 8, 9, or 10 of the substitutions to increase affinity to FCGR3A.

The polypeptide of a high-affinity Fc-polypeptide of the invention specifically binds to and agonizes human DR4 and/or human DR5 thereby inducing apoptosis in sensitive human cancer cells. Methods of screening Fc-polypeptides for the ability to agonize DR5 and/or DR4 are known to those of ordinary skill in the art. The polypeptide of an Fc-polypeptide of the invention can be obtained from a number of sources such as by screening a phage library for peptides that specifically bind to the DR4 and/or DR5 target. Methods of making and screening peptide libraries are well known in the art. Peptides having the desired specific binding properties can be covalently attached, directly or indirectly (i.e., via a linker), to an Fc to yield the Fc-polypeptide. In some embodiments the Fc is a human IgG1 Fc. In other embodiments the polypeptide is the antigen binding site of an anti-DR4 and/or anti-DR5 antibody comprising complementary determining regions (CDR): CDR1, CDR2, and/or CDR3 of the antibody. Conveniently, the variable heavy and variable light chains of an immunoglobulin, such as an antibody that specifically binds to human DR4 and/or human DR5, can be utilized in an Fc-polypeptide of the invention. Thus, in some embodiments the Fc-polypeptide is itself a bivalent IgG1 antibody, such as a fully human monoclonal antibody, that specifically binds to human DR5 and/or human DR4. In some embodiments, the polypeptide of the Fc-polypeptide is a scFv (single-chain Fv), Fab or F(ab′)₂ fragment of an antibody that specifically binds to human DR4 and/or human DR5, or a peptide aptamer that specifically binds to human DR4 and/or human DR5. Representative Fc-polypeptides that can be modified according to the methods of the invention to yield a high affinity Fc-polypeptide with enhanced anti-cancer activity include the anti-DR5 agonist antibodies conatumumab (Amgen Inc.), lexatumumab (Human Genome Sciences, Inc.), drozitumab (Genentech, Inc.), and, tigatuzumab (Daiichi Sankyo, Inc.), and the anti-DR4 agonist antibody mapatumumab (Human Genome Sciences, Inc.). In another embodiment, the polypeptide of an Fc-polypeptide of the invention is human TRAIL (TNF-Receptor Apoptosis Inducing Ligand) ligand. In a particular embodiment the Fc-polypeptide is a bivalent Fc-polypeptide wherein the polypeptide is a human TRAIL ligand.

The Fc of an Fc-polypeptide of the invention can be obtained by a variety of methods well known in the art including, but not limited to, recombinant expression methods, solid-phase peptide synthetic methods, isolated from natural sources such as human immunoglobulins, or combinations of these methods. In some embodiments, the Fc is a human IgG1. In certain embodiments, the Fc of one isotype is converted to a different isotype by isotype switching. Methods of isotype switching include, but are not limited to, direct recombinant techniques and cell-cell fusion techniques (see e.g., U.S. Pat. No. 5,916,771), among others. In certain embodiments, an Fc is converted from a human IgG2, IgG3, or IgG4 subclass to a human IgG1 subclass. Those of skill in the art will recognize that in order to optimize solubility, manufacturability, stability, and other factors relevant to the manufacture of biopharmaceuticals, several amino acid residues of a native human IgG1 can be modified yet still be within the definition of human IgGl. Generally, no more than a total of up to 15 residues are deleted, added, and or substituted and often no more than 10, 9, 8, 7, 6, 5, 4, 3, 2, or 1. The Fc of an Fc-polypeptide can, however, be linked directly or indirectly with labels, toxins, drugs, tissue-specific binding agents, and the like, to enhance the pharmacokinetic or pharmacodynamic properties of the Fc-polypeptide.

In some embodiments, two or more Fc-polypeptides can be covalently bonded to one another, such as by cysteine-cysteine disulfide bonds, to create a bivalent (i.e., two antigen binding sites), trivalent, or higher order structures of Fc-polypeptides. A bivalent Fc-polypeptide, such as an antibody, can be monospecific and specifically bind to a single epitope of the target, or bispecific such that it specifically binds to two unique epitopes on the same target (e.g., human DR5 or human DR4) or two unique epitopes of differing targets (e.g., human DR4 and human DR5). In additional embodiments, two or more polypeptides that specifically bind to human DR4 and/or human DR5 are covalently linked to a single Fc to form an Fc-polypeptide of the invention. Thus, in some embodiments, 2, 3, or 4 of such polypeptides are covalently linked to a single Fc. Such Fc-polypeptides can be dimerized (by, for example, disulfide bonding between Fc chains to form a bivalent Fc-polypeptide), trimerized, etc. The polypeptide can be directly or indirectly attached to an Fc at or near the N-or C-terminus of the Fc or at a residue within the Fc. In other embodiments, a second or additional polypeptide that specifically binds to human DR4 and/or human DR5 is covalently linked to a polypeptide that itself is covalently linked to the Fc. Thus, Fc-polypeptides can comprise multiple polypeptides covalently linked, directly or indirectly, to the Fc or to a polypeptide that is itself covalently attached directly or indirectly to the Fc.

In certain embodiments, an Fc-polypeptide (e.g., an antibody) of the invention can be constructed using recombinant methods. Therefore, another aspect of the invention is a polynucleotide encoding an Fc-polypeptide of the invention. In another aspect the present invention comprises an expression vector comprising the polynucleotide encoding an Fc-polypeptide. In certain embodiments, the expression vectors comprise control sequences (e.g., promoters, enhancers) that are operably linked to a polynucleotide encoding an Fc-polypeptide so as support expression in a suitable host cell. In certain embodiments, the expression vector also comprises polynucleotide sequences that allow chromosome-independent replication in the host cell. Exemplary vectors include, but are not limited to, plasmids, cosmids, and YACS. In yet another aspect, the invention comprises a host cell comprising the expression vector of the invention. Methods of transfecting suitable host cells (e.g., CHO cells) with the expression vector of the invention and culturing the transfected host cells under conditions suitable for expression of an Fc-polypeptide are known in the art. The transfection procedure used may depend upon the host to be transformed. Certain methods for introduction of heterologous polynucleotides into mammalian cells are known in the art and include, but are not limited to, dextran-mediated transfection, calcium phosphate precipitation, polybrene mediated transfection, protoplast fusion, electroporation, encapsulation of the polynucleotide(s) in liposomes, and direct microinjection of the DNA into nuclei. Certain mammalian cell lines available as hosts for expression are known in the art and include, but are not limited to, many immortalized cell lines available from the American Type Culture Collection (ATCC), including but not limited to Chinese hamster ovary (CHO) cells, E5 cells, baby hamster kidney (BHK) cells, monkey kidney cells (COS), human hepatocellular carcinoma cells (e.g., Hep G2), and a number of other cell lines. In certain embodiments, cell lines may be selected through determining which cell lines have high expression levels and produce Fc-polypeptides with desired antigen binding properties.

### Therapeutic Applications

An Fc-polypeptide of the invention (a “therapeutic composition”) can be used to inhibit growth of human cancer cells as a monotherapy (i.e., as a single agent), in combination with at least one chemotherapeutic agent (i.e., a combination therapy), and/or in combination with radiation therapy. An effective amount of a therapeutic composition is administered to inhibit, halt, or reverse progression of cancers that are sensitive to DR4 and/or DR5 mediated apoptosis. Human cancer cells can be treated in vivo, or ex vivo. In ex vivo treatment of a human patient, tissue or fluids containing cancer cells are treated outside the body and then the tissue or fluids are reintroduced back into the patient. In some embodiments, the cancer is treated in a human patient in vivo by administration of the therapeutic composition into the patient. Thus, the present invention provides ex vivo and in vivo methods to inhibit, halt, or reverse progression of the tumor, or otherwise result in a statistically significant increase in progression-free survival (i.e., the length of time during and after treatment in which a patient is living with pancreatic cancer that does not get worse), or overall survival (also called “survival rate”; i.e., the percentage of people in a study or treatment group who are alive for a certain period of time after they were diagnosed with or treated for cancer) relative to treatment with a control.

The cancers which can be treated by the methods of the invention include, but are not limited to, liver cancer, brain cancer, renal cancer, breast cancer, pancreatic cancer (adenocarcinoma), colorectal cancer, lung cancer (small cell lung cancer and non-small-cell lung cancer), spleen cancer, cancer of the thymus or blood cells (i.e., leukemia), prostate cancer, testicular cancer, ovarian cancer, uterine cancer, gastric carcinoma, head and neck squamous cell carcinoma, melanoma, and lymphoma. In some embodiments the cancer is non-small cell lung cancer (NSCLC).

### Pharmaceutical Compositions

The therapeutic compositions of the invention (i.e., Fc-polypeptide) can each be administered alone as a monotherapy or as a combination therapy, i.e., combined with other agents (e.g., anti-angiogenic agents, chemotherapeutic agents, radiation therapy). Exemplary chemotherapeutic agents include, but are not limited to, adriamycin, doxorubicin, 5-fluorouracil, cytosine arabinoside, cyclophosphamide, thiotepa, docetaxel, busulfan, cytoxin, taxol, paclitaxel, methotrexate, gemcitabine, cisplatin, melphalan, vinblastine, bleomycin, etoposide, ifosfamide, mitomycin C, mitoxantrone, vincristine, vinorelbine, carboplatin, teniposide, daunomycin, carminomycin, aminopterin, dactinomycin, mitomycins, esperamicins, melphalan and other related nitrogen mustards.

A chemotherapeutic agent of the present invention can be administered prior to and/or subsequent to (collectively, “sequential treatment”), and/or simultaneously with (“concurrent treatment”) a specific binding agent of the present invention. Sequential treatment (such as pretreatment, post-treatment, or overlapping treatment) of the combination, also includes regimens in which the drugs are alternated, or wherein one component is administered long-term and the other(s) are administered intermittently. Components of the combination may be administered in the same or in separate compositions, and by the same or different routes of administration.

Exemplary cancer therapies, which may be co-administered with a therapeutic composition of the invention include, HERCEPTIN™ (trastuzumab), which may be used to treat breast cancer and other forms of cancer; RITUXAN™ (rituximab), ZEVALIN™ (ibritumomab tiuxetan), and LYMPHOCIDE™ (epratuzumab), which may be used to treat non-Hodgkin's lymphoma and other forms of cancer; GLEEVEC™ (imatinib mesylate), which may be used to treat chronic myeloid leukemia and gastrointestinal stromal tumors; and BEXXAR™ (tositumomab), which may be used for treatment of non-Hodgkin's lymphoma. Certain exemplary antibodies also include ERBITUX™; VECTIBIX™, IMC-C225; IRESSA™ (gefitinib); TARCEVA™ (ertinolib); KDR (kinase domain receptor) inhibitors; anti VEGF antibodies and antagonists (e.g., AVASTIN™ and VEGF-traps); anti-VEGF (vascular endothelial growth factor) receptor antibodies, peptibodies, and antigen binding regions; anti-Ang-1 and Ang-2 antibodies, peptibodies (e.g., AMG 386, Amgen Inc), and antigen binding regions; antibodies to Tie-2 and other Ang-1 and Ang-2 receptors; Tie-2 ligands; antibodies against Tie-2 kinase inhibitors; and CAMPATH™, (alemtuzumab).

### Pharmaceutical Formulation

A pharmaceutical composition comprising a therapeutic composition of the present invention may contain formulation materials for modifying, maintaining or preserving, for example, the pH, osmolarity, viscosity, clarity, color, isotonicity, odor, sterility, stability, rate of dissolution or release, adsorption, or penetration of the composition. The primary vehicle or carrier in a pharmaceutical composition may be either aqueous or non-aqueous in nature. For example, a suitable vehicle or carrier may be water for injection or physiological saline, possibly supplemented with other materials common in compositions for parenteral administration. Neutral buffered saline or saline mixed with serum albumin are further exemplary vehicles. Other exemplary pharmaceutical compositions comprise Tris buffer of about pH 7.0-8.5, or acetate buffer of about pH 4.0-5.5, which may further include sorbitol or a suitable substitute therefore. In one embodiment of the present invention, binding agent compositions may be prepared for storage by mixing the selected composition having the desired degree of purity with optional formulation agents (Remington's Pharmaceutical Sciences, supra) in the form of a lyophilized cake or an aqueous solution. Further, the binding agent product may be formulated as a lyophilizate using appropriate excipients such as sucrose.

The formulation components are present in concentrations that are acceptable to the site of administration. For example, buffers are used to maintain the composition at physiological pH or at slightly lower pH, typically within a pH range of from about 5 to about 8. A particularly suitable vehicle for parenteral administration is sterile distilled water in which a binding agent is formulated as a sterile, isotonic solution, properly preserved. Yet another preparation can involve the formulation of the desired molecule with an agent, such as injectable microspheres, bio-erodible particles, polymeric compounds (polylactic acid, polyglycolic acid), beads, or liposomes, that provide for the controlled or sustained release of the product which may then be delivered via a depot injection.

In another aspect, pharmaceutical formulations suitable for parenteral administration may be formulated in aqueous solutions, preferably in physiologically compatible buffers such as Hanks' solution, ringer's solution, or physiologically buffered saline. Aqueous injection suspensions may contain substances that increase the viscosity of the suspension, such as sodium carboxymethyl cellulose, sorbitol, or dextran. Additional pharmaceutical compositions will be evident to those skilled in the art, including formulations involving binding agent molecules in sustained- or controlled-delivery formulations. Techniques for formulating a variety of other sustained- or controlled-delivery means, such as liposome carriers, bio-erodible microparticles or porous beads and depot injections, are also known to those skilled in the art. The pharmaceutical composition to be used for in vivo administration typically must be sterile. This may be accomplished by filtration through sterile filtration membranes. Where the composition is lyophilized, sterilization using this method may be conducted either prior to or following lyophilization and reconstitution. The composition for parenteral administration may be stored in lyophilized form or in solution. In addition, parenteral compositions generally are placed into a container having a sterile access port, for example, an intravenous solution bag or vial having a stopper pierceable by a hypodermic injection needle.

Once the pharmaceutical composition has been formulated, it may be stored in sterile vials as a solution, suspension, gel, emulsion, solid, or a dehydrated or lyophilized powder. Such formulations may be stored either in a ready-to-use form or in a form (e.g., lyophilized) requiring reconstitution prior to administration. An effective amount of a pharmaceutical composition to be employed therapeutically will depend, for example, upon the therapeutic context and objectives. One skilled in the art will appreciate that the appropriate dosage levels for treatment will thus vary depending, in part, upon the molecule delivered, the indication for which the binding agent molecule is being used, the route of administration, and the size (body weight, body surface or organ size) and condition (the age and general health) of the patient. Accordingly, the clinician may titer the dosage and modify the route of administration to obtain the optimal therapeutic effect. A typical dosage may range from about 0.1 mg/kg to up to about 50 mg/kg or more, depending on the factors mentioned above. In some embodiments, the dosage can be 1, 3, 5, 10, 15, 20, 25, or 30 mg/kg.

For any compound, the therapeutically effective dose can be estimated initially either in cell culture assays or in animal models such as mice, rats, rabbits, dogs, pigs, or monkeys. An animal model may also be used to determine the appropriate concentration range and route of administration. Such information can then be used to determine useful doses and routes for administration in humans. The exact dosage will be determined in light of factors related to the subject requiring treatment. Dosage and administration are adjusted to provide sufficient levels of the active compound or to maintain the desired effect. Factors that may be taken into account include the severity of the disease state, the general health of the subject, the age, weight, and gender of the subject, time and frequency of administration, drug combination(s), reaction sensitivities, and response to therapy. Long-acting pharmaceutical compositions may be administered every 3 to 4 days, every week, or biweekly depending on the half-life and clearance rate of the particular formulation. The frequency of dosing will depend upon the pharmacokinetic parameters of the binding agent molecule in the formulation used. Typically, a composition is administered until a dosage is reached that achieves the desired effect. The composition may therefore be administered as a single dose, or as multiple doses (at the same or different concentrations/dosages) over time, or as a continuous infusion. Further refinement of the appropriate dosage is routinely made. Appropriate dosages may be ascertained through use of appropriate dose-response data.

### FCGR3A Genotyping

The present invention provides a method of identifying a human patient (or patients) whom is more likely to obtain a clinical benefit from treatment with an agonistic high-affinity Fc-polypeptide of the present invention (relative to a control) as evidenced by a statistically significant increased response in progression-free survival and/or overall survival. Such patients are heterozygous (F158/V158) or homozygous (F158/F158) for the F158 polymorphism of FCGR3A. Patients can be stratified on the basis of this allelic difference and those identified as having one or two copies of the allele coding for the F158 allele of FCGR3A can then be treated by the therapeutic composition herein disclosed. Identifying a patient having a V158 and F158 polymorphism can be achieved employing analytical methods known to those of skill in the art such as PCR based methods (Leppers-van de Straat et al., J. Immunological Methods, 242: 127-132 (2000)). Conveniently, a clinician can identify such patients using the services of third party laboratories to carry out such methods. Kits for identifying patients having 0, 1, or 2 copies of the V158 or F158 allele of FCGR3A in cancer patients are also within the scope of the present invention. Such kits can optionally contain written instructions identifying the allelic forms of patients who are more likely to respond to a high-affinity Fc therapeutic composition (i.e., F158/V158 and F158/F158 patients).

In another aspect, the present invention relates to compositions and methods for real-time PCR (polymerase chain reaction) genotyping of human genomic DNA for FCGR3A polymorphisms, F158 and V158, using an allelic discrimination assay. Methods of isolating and purifying human genomic DNA are known in the art. In the method PCR primers specifically amplify a region containing the single nucleotide polymorphism (SNP) of FCGR3A commonly referred to as F158V (SNP ID: rs396991). In the method the forward primer comprises the sequence 5′-TTCCAAAAGCCACACTCAAACAC-3′ (SEQ ID NO: 1) while the reverse primer comprises: 5′-TGGTGATGTTCACAGTCTCTGAAGA-3′(SEQ ID NO: 2). The forward primer specifically anneals upstream of the SNP in a region with a single nucleotide difference between the FCGR3A and FCGR2A genes. Furthermore, a mismatch is incorporated into the forward primer three nucleotides from the 3′ end to maximize the discriminatory power of the 3′ end. The reverse primer anneals downstream of the SNP, in a region that is exactly the same sequence in both FCGR3A and FCGR2A. Therefore, the assay relies on the forward primer for gene specificity. The primers amplify a 93 base pair amplicon.

Following PCR amplification a pair of dual-labeled probes determines the genotype of the donor. The probes are specifically annealed, under specific annealing conditions, to the internal region of the amplicon with the SNP located near the center of the hybridization region. Methods and compositions for specific annealing are known to those of skill in the art. One probe is specific for each of the two measured SNP alleles. In some embodiments, the probe is labeled. In one embodiment, one probe is labeled at the 5′ end with the fluorescence reporter dye Fluorescein amidite (FAM). FAM can be obtained commercially from a number of sources. See, e.g., Glen Research, Sterling, Va., USA). The other probe is labeled with a different reporter dye. In one embodiment the probe is labeled with Yakima Yellow (YAK). See, Eurogentee, San Diego, Calif., USA). Both probes can be modified at the 3′ end with a quencher, such as Black-Hole quencher (BHQ). BHQ is available commercially from a number of sources. See, e.g., Glen Research, Sterling, Va., USA). Thus, in some embodiments, one probe is specific for V158 and comprises the sequence: 5′-<FAM>TTACTCCCAAAAAGCCCCCTGCA-3′<BHQ>(SEQ ID NO: 3) and the other probe is specific for the F158 allele and comprises the sequence: 5′<YAK>TACTCCCAACAAGCCCCCTGCA-3′<BHQ>(SEQ ID NO: 4).

When the probe is intact, the fluorescence of the reporter dye is quenched by the proximity of the quencher dye. During the extension phase of each PCR cycle, DNA polymerase cleaves the annealed probe, releasing the reporter dye from the probe, resulting in an increase in fluorescence. The probes compete for hybridization during the PCR cycling, and fluorescence is generated only from the probe complementary to the SNP allele present in the DNA. In the case of heterozygosity, fluorescence is generated by both probes. The fluorescence levels are measured after sufficient number of amplification cycles, such as 40 cycles of PCR. Thus, this SNP assay classifies human genomic DNA samples as F158/F158(homozygote), V158/V158(homozygote), and F158/V158(heterozygote).

The above listings are by way of example only, and do not preclude the use of other compounds or treatments which can be used concurrently with the compounds described herein that are known by those skilled in the art or that could be arrived at by those skilled in the art using the guidelines set forth in this specification.

### EXAMPLE 1

Example 1 describes a Phase 1b/2 Study of conatumumab in Combination With Paclitaxel and Carboplatin for the First-Line Treatment of Advanced Non-Small Cell Lung Cancer

The primary objective is to estimate the efficacy of conatumumab (AMG 655) as assessed by progression-free survival time (at two doses selected in phase 1b: 3 mg/kg and 15 mg/kg) in combination with paclitaxel/carboplatin.
Inclusion Criteria for the study included:
Histologically or cytologically confirmed non-small cell lung cancer.
Subjects must have advanced non-small cell lung cancer defined as stage IIIB with malignant pleural effusion or stage IV or recurrent disease.
Planning to receive up to 6 cycles of chemotherapy
Eastern Cooperative Oncology Group (ECOG) score of 0 or 1 Demographic
Men or women >18 years of age
Adequate Hematological, renal, hepatic and coagulation function

The doses of conatumumab for phase 2 were determined during a phase lb portion of the study. The phase 2 portion of this study is a multi-center, randomized, double-blind, placebo-controlled study with a planned total sample size of 150 subjects. Subjects were randomized at a 1:1:1 ratio to 1 of 3 treatment arms: Arm 1: Paclitaxel/carboplatin plus 15 mg/kg conatumumab
Arm 2: Paclitaxel/carboplatin plus 3 mg/kg conatumumab
Arm 3: Paclitaxel/carboplatin plus placebo
Randomization was stratified by Eastern Cooperative Oncology Group Performance
Status (ECOG) 0 or 1) and disease stage (Mb or IV/recurrent).

Following enrollment, paclitaxel (200 mg/m2) and carboplatin (AUC (area under curve)=6 mg/mL-minute) in combination with conatumumab (Arm 1 and Arm 2) or placebo (Arm 3) were administered on day 1 of each 21 day cycle for up to a maximum 6 cycles. Subjects who completed up to 6 cycles of paclitaxel/carboplatin or who discontinued paclitaxel/carboplatin due to chemotherapy-related toxicity continued to receive conatumumab or placebo monotherapy until disease progression, drug (conatumumab or placebo) intolerability, withdrawal of consent, or until 30 months from the first administration of study treatment, whichever occurred first. Subjects who discontinued treatment with conatumumab or placebo, due to suspected drug intolerability, were withdrawn from all study treatment.

After the last administration of all protocol specified treatments each subject was scheduled to have a safety follow up visit 30 days (+3 days) later. Subjects then entered long-term follow-up during which survival status was checked every 3 months (±2 weeks).

Radiological imaging to evaluate tumor response was performed every six weeks (±7 days) from study day 1 independent of the treatment cycle until documented disease progression (determined clinically or radiologically per modified RECIST by the treating investigator) . In general, subjects with symptoms suggestive of disease progression (clinical progression) were also evaluated radiologically to assess disease status at the time the symptoms occur. Any subject who discontinued study treatment prior to disease progression or death continued to have radiological imaging performed every six weeks (±7 days) during the long term follow-up period to assess disease status until disease progression, start of a new treatment, death, withdrawal of consent, administrative decision or the end of the study, whichever is earlier. The primary analysis was performed when 120 subjects had documented investigator assessed disease progression or death.

Results showed no improvement in PFS (progression free survival). However, a trend suggesting improvement in OS (overall survival) curves emerged after approximately 7 months; shortly after initiation of the monotherapy (conatumumab) phase of treatment. Patients were in monotherapy phase, after completion of 6 cycles of chemotherapy+conatumumab or placebo (approximately 4.2 months) Monotherapy occurred in 51% of subjects post combination chemotherapy. When patient outcome was adjusted for gender, ECOG, age, & histology (squamous) and associated with FCGR3A genotype a dose-dependent response was observed for patients heterozygous and homozygous for V158. As indicated, the survival trend associated with the F158N158 or V158N158 genotype of the FCGR3A polymorphism yielded a hazard ration (HR) of 0.72 as compared to 1.37 for F158 homozygous patients. Further, the HR of FV (F158/V158) and VV (V158/V158) patients shows a dose-response effect with a HR of 0.63 for the dose of 15 mg/kg as opposed to 0.85 at the dose of 3 mg/kg.

Table 1 below shows the hazard ratio (HR) and 95% confidence intervals (95% CI).

TABLE 1

| | HR | HR | |
|---|---|---|---|
| | (3 mg) | (15 mg) | Combined |
| FV + VV | 0.85 | 0.63 | 0.72 |
| (95% CI) | (0.45, 1.58) | (0.34, 1.19) | (0.43, 1.23) |
| FF | 1.35 | 1.40 | 1.37 |
| | (0.59, 3.10) | (0.60, 3.24) | (0.66, 2.86) |

What is claimed is:

1. A high-affinity Fc-polypeptide that specifically binds to and agonizes human DR4 or DRS.

2. The Fc-polypeptide of claim 1, wherein said Fc-polypeptide is an antibody.

3. The Fc-polypeptide of claim 2, wherein said antibody is an anti-DR5 antibody.

4. The Fc-polypeptide of claim 3, wherein said antibody is a fully human IgG1 antibody.

5. The Fc-polypeptide of claim 4, wherein said antibody is afucosylated.

6. The Fc-polypeptide of claim 4, wherein said antibody comprises an amino acid at position 332 of the Fc numbered according to the EU index, wherein said amino acid increases affinity of said Fc-polypeptide to human FCGR3A.

7. A method of inhibiting cancer growth in a human patient, comprising administering to said patient a therapeutically effective amount of the high-affinity Fc-polypeptide of claim 4.

8. The method of claim 7, wherein said cancer is non-small cell lung cancer (NSCLC).

9. The method of claim 8, wherein said high-affinity Fc-polypeptide is administered as a monotherapy.

10. The method of claim 8, wherein said human patient is heterozygous or homozygous for allele F158 of FCGR3A.

11. A method of inhibiting growth of a human cancer cell comprising administering to said cell an effective amount of the high-affinity Fc-polypeptide of claim 4.

12. The method of claim 11, wherein the cancer cell is non-small cell lung cancer.

13. A method of inhibiting growth of a human cancer cell comprising administering to said cell an effective amount of the high-affinity Fc-polypeptide of claim 5.

14. The method of claim 13, wherein the cancer cell is non-small cell lung cancer.

15. A method of inhibiting growth of a human cancer cell comprising administering to said cell an effective amount of the high-affinity Fc-polypeptide of claim 6.

16. The method of claim 15, wherein the cancer cell is non-small cell lung cancer.

17. A method for selecting a cancer patient for treatment with a high-affinity Fc-polypeptide that specifically binds to and agonizes human DR4 or DR5, wherein said patient has at least one F158 allele of FCGR3A.

18. The method of claim 17, wherein said patient has two F158 FCGR3A alleles.

19. The method of claim 18, wherein said patient has non-small cell lung cancer.

20. A method of genotyping for a F158V FCGR3A polymorphism in a human genomic DNA sample, comprising amplifying a region comprising said FCGR3A polymorphism in said genomic DNA sample using a forward primer of SEQ ID NO: 1 and a reverse primer of SEQ ID NO: 2, and genotyping said DNA sample for homozygosity or heterozygosity of said F158V polymorphism with a probe of SEQ ID NO: 3 and SEQ ID NO: 4.
